(19) United States
(12) Patent Application Publication
(10) Pub. No.: US 2002/0161395 A1
(43) Pub. Date: Oct. 31, 2002

(54) Guide wire apparatus for prevention of distal atheroembolization

(76) Inventors: Nareak Douk, Lowell, MA (US); Nasser Rafiee, Andover, MA (US); Peter G. Strickler, Tewksbury, MA (US); Michael S. Noone, Londonberry, NH (US); Mukund R. Patel, San Jose, CA (US); Jeffrey A. Nool, San Jose, CA (US); Celso J. Bagaoisan, Union City, CA (US); Hung V. Ha, San Jose, CA (US)

Correspondence Address: MEDTRONIC AVE, INC., 3576 UNOCAL PLACE, SANTA ROSA, CA, 95403, US

(21) Appl. No.: 10/116,238

(22) Filed: Apr. 4, 2002

Related U.S. Application Data
(63) Continuation-in-part of application No. 09/922,996, filed on Aug. 1, 2001.
(63) Continuation-in-part of application No. 09/918,441, filed on Jul. 27, 2001.
Application No. 09/918,441 is a continuation-in-part of application No. 09/824,832, filed on Apr. 3, 2001.

(51) Int. Cl. A61M 29/00

(52) U.S. Cl. 606/200

(57) ABSTRACT

A guide wire apparatus for prevention of distal atheroembolization during percutaneous catheter intervention procedures, such as angioplasty or stent deployment. An expandable protection element, such as a self-expanding or self-collapsing filter or occluder, is mountable adjacent the distal end of a core wire, which can guide a therapeutic catheter. Relative displacement of the ends of the protection element causes transformation of the protection element between a closed configuration and an expanded configuration that spans the vessel to be treated. An operator rod, which may be a hollow rod or an interventional catheter, may be slidably disposed over the core wire to engage with and either push or pull the proximal end of the protection element to cause transformation thereof.

### CROSS-REFERENCE TO RELATED APPLICATION

This patent application is a continuation-in-part of U.S. Patent application Ser. Nos. 09/922,996 filed Aug. 1, 2001 and 09/918,441 filed Jul. 27, 2001, which are both continuations-in-part of U.S. Patent application Ser. No. 09/824,832 to Douk et al. filed Apr. 3, 2001 entitled “Temporary Intraluminal Filter Guidewire and Methods of Use.”

### FIELD OF THE INVENTION

The present invention relates generally to intraluminal devices for trapping particulate in the vessels of a patient. More particularly, the invention relates to protection elements for trapping atheroemboli in a blood vessel during an interventional vascular treatment and then removing the trapped atheroemboli from the patient after completion of the treatment. Furthermore, the invention concerns a filter or an occluder mounted on a guide wire that can also be used to direct an interventional catheter to a treatment site within a patient.

### BACKGROUND OF THE INVENTION

A variety of treatments exists for dilating or removing atherosclerotic plaque in blood vessels. The use of an angioplasty balloon catheter is common in the art as a minimally invasive treatment to enlarge a stenotic or diseased blood vessel. When applied to the vessels of the heart, this treatment is known as percutaneous transluminal coronary angioplasty, or PTCA. To provide radial support to the treated vessel in order to prolong the positive effects of PTCA, a stent may be implanted in conjunction with the procedure.

Thrombectomy is a minimally invasive technique for removal of an entire thrombosis or a sufficient portion of the thrombosis to enlarge the stenotic or diseased blood vessel and may be accomplished instead of a PTCA procedure. Atherectomy is another well-known minimally invasive procedure that mechanically cuts or abrades a stenosis within the diseased portion of the vessel. Alternatively, ablation therapies use laser or RF signals to superheat or vaporize the thrombus within the vessel. Atheroemboli loosened during such procedures may be removed from the patient through the catheter.

During each of these procedures, there is a risk that atheroemboli dislodged by the procedure will migrate through the circulatory system and cause ischaemic events, such as infarction or stroke. Thus, practitioners have approached prevention of escaped atheroemboli through use of occlusion devices, filters, lysing, and aspiration techniques. For example, it is known to remove the atheroembolic material by suction through an aspiration lumen in the treatment catheter or by capturing atheroemboli in a filter or occlusion device positioned distal of the treatment area.

### SUMMARY OF THE INVENTION

A guide wire apparatus is provided for prevention of distal atheroembolization during percutaneous catheter intervention procedures, such as angioplasty or stent deployment. An expandable protection element, such as a filter or an occluder, is mountable adjacent the distal end of a core wire, which can guide a therapeutic catheter. As distinguished from filter guide wire embodiments of the invention, occluder guide wires are typically used to temporarily obstruct fluid flow through the vessel being treated. Any atheroembolic debris trapped upstream of the occluder element may be aspirated using a separate catheter, with or without irrigation of the area. Relative displacement of the protection element ends causes transformation of the protection element between a closed configuration and an expanded configuration that spans the vessel to be treated. An operator rod, which may be a hollow rod or an interventional catheter, may be slidably disposed over the core wire to engage with and either push or pull the proximal end of the protection element to cause transformation thereof.

### BRIEF DESCRIPTION OF THE DRAWINGS

Features, aspects and advantages of the present invention will become better understood with reference to the following description, appended claims, and accompanying drawings where:

FIG. 1 is an illustration of a guide wire apparatus in accordance with the invention deployed within a blood vessel;

FIG. 2 is an illustration of a guide wire apparatus in accordance with the invention deployed within a portion of the coronary arterial anatomy;

FIG. 3 is an illustration of a prior art expandable mesh device, shown with the mesh in a collapsed configuration;

FIG. 4 is an illustration of a prior art expandable mesh device, shown with the mesh in a deployed configuration;

FIG. 5 is a longitudinal sectional view of a first embodiment in accordance with the invention;

FIG. 6 is a longitudinal sectional view of a second embodiment in accordance with the invention;

FIGS. 7-8 are longitudinal sectional illustrations of an operator rod distal portion in accordance with the second embodiment the invention;

FIGS. 9-10 are elevation views of alternate operator rod gripping elements in accordance with the second embodiment of the invention;

FIGS. 11-12 are elevation views of alternate operator rod locking mechanisms in accordance with the second embodiment the invention;

FIG. 13 is an elevation view, in partial section, of another alternate operator rod locking mechanism in accordance with the second embodiment of the invention;

FIG. 14 is a longitudinal sectional view of a portion of the third guide wire apparatus embodiment in accordance with the invention;

FIG. 15 is a partially schematic elevation view of a third embodiment in accordance with the invention;

FIG. 16 is an elevation view, in partial section, of a tubular actuator in accordance with the third embodiment of the invention;

FIG. 17 is a longitudinal sectional view of a fourth embodiment of a protection element for use with a guide wire apparatus in accordance with the invention, shown in a collapsed configuration;

FIG. 18 is a longitudinal sectional view of the fourth embodiment of a protection element for use with a guide wire apparatus in accordance with the invention, shown in an expanded configuration;

FIG. 19 is an elevation view of the fourth embodiment in accordance with the invention, shown in a collapsed configuration on a guide wire; and

FIG. 20 is an elevation view of the fourth embodiment in accordance with the invention, shown in an expanded configuration on a guide wire.

### DETAILED DESCRIPTION OF THE INVENTION

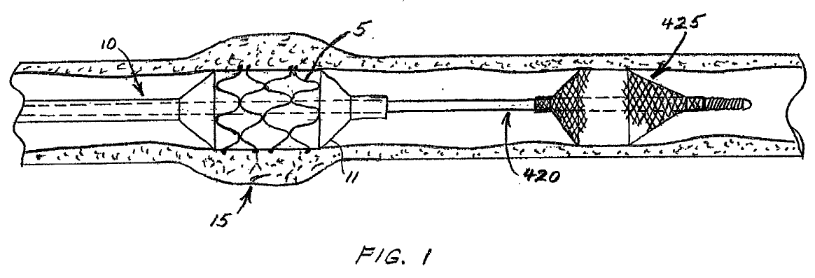

The present invention is a guide wire apparatus for use in minimally invasive procedures. While the following description of the invention relates to vascular interventions, it is to be understood that the invention is applicable to other procedures where the practitioner desires to capture embolic material that may be dislodged during the procedure. Intravascular procedures such as PTCA or stent deployment are often preferable to more invasive surgical techniques in the treatment of vascular narrowings, called stenoses or lesions. With reference to FIGS. 1 and 2, deployment of balloon expandable stent 5 is accomplished by threading catheter 10 through the vascular system of the patient until stent 5 is located within a stenosis at predetermined treatment site 15. Once positioned, balloon 11 of catheter 10 is inflated to expand stent 5 against the vascular wall to maintain the opening. Stent deployment can be performed following treatments such as angioplasty, or during initial balloon dilation of the treatment site, which is referred to as primary or direct stenting.

Catheter 10 is typically guided to treatment site 15 by a guide wire. In cases where the target stenosis is located in tortuous vessels that are remote from the vascular access point, such as coronary arteries 17 shown in FIG. 2, a steerable guide wire is commonly used. According to the present invention, a guide wire apparatus generally guides catheter 10 to treatment site 15 and includes a distally disposed protection element to collect atheroembolic debris that may be generated during the procedure. Various embodiments of the invention will be described as either filter guide wires or occluder guide wires. However, it is to be understood that filters and occluders are interchangeable types of protection elements among the inventive structures disclosed. The invention is directed to atheroembolic protection elements wherein relative movement of the ends of the protection element either causes or accompanies transformation of the element between a collapsed configuration and an expanded, or deployed configuration. Such transformation may be impelled by external mechanical means or by self-shaping memory (either self-expanding or self-collapsing) within the protection element itself. The protection element may be self-expanding, meaning that it has a mechanical memory to return to the expanded, or deployed configuration. Such mechanical memory can be imparted to the metal comprising the element by thermal treatment to achieve a spring temper in stainless steel, for example, or to set a shape memory in a susceptible metal alloy such as a nickel-titanium (nitinol) alloy.

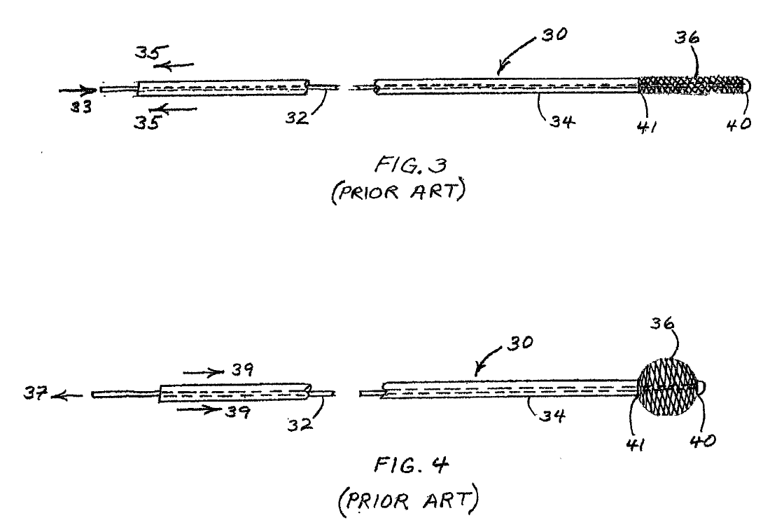

A guide wire apparatus in accordance with the invention includes a distally disposed protection element, such as filter 25, shown in FIG. 6, or occluder 425, shown in FIG. 5. Such protection elements may comprise a tube formed by braided filaments 426, which can form a porous filter. Alternatively, braided filaments 426 may be replaced with expandable struts, and either type of support structure can be covered with an elastic membrane. A porous membrane can be used to make a filter element, and nonporous membrane can be used to make an occluder. Optionally, adding radiopaque markers, such as those shown on filter ends 27, 29 in FIG. 6, can aid in fluoroscopic observation of a protection element during manipulation thereof. Alternatively, to enhance visualization under fluoroscopy, at least one of braided filaments 426 maybe a wire having enhanced radiopacity compared to conventional non-radiopaque wires suitable for braiding a protection element. At least the majority of braiding wires forming a protection element should be capable of being heat set into the desired shape, and such wires should also have sufficient elastic properties to provide the desired self-expanding or self-collapsing features. Stainless steel and nitinol monofilaments are suitable for braiding a protection element. A braiding wire having enhanced radiopacity may be made of, or coated with, a radiopaque metal such as gold, platinum, tungsten, alloys thereof, or other biocompatible metals that, compared with stainless steel or nitinol, have a relatively high X-ray attenuation coefficient. One or more filaments having enhanced radiopacity may be inter-woven with non-radiopaque wires, or all wires comprising a protection element may have the same enhanced radiopacity.

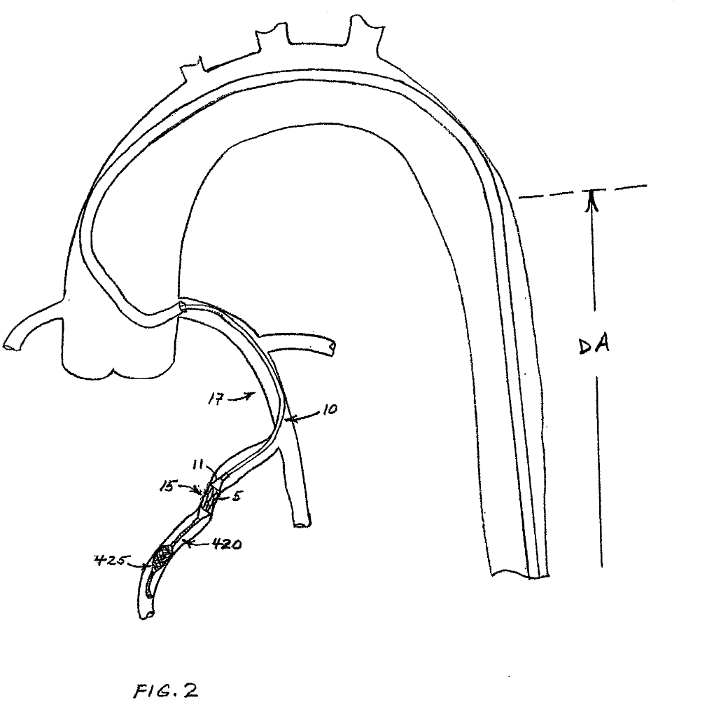

During introduction and withdrawal of a guide wire apparatus of the instant invention, the collapsed protection element does not require a control sheath to slidingly envelope the protection element. Thus, this type of device is sometimes termed “sheathless.” Known types of sheathless vascular protection devices are operated by a push-pull mechanism that is also typical of other expandable braid devices, as shown in FIGS. 3 and 4. Prior art expandable mesh device 30 includes core wire 32 and hollow shaft 34 movably disposed there about. Tubular mesh, or braid 36 surrounds core wire 32 and has a braid distal end fixed to core wire distal end 40 and a braid proximal end fixed to shaft distal end 41. To expand braid 36, core wire 32 is pulled and shaft 34 is pushed, as shown by arrows 37 and 39 respectively in FIG. 4. The relative displacement of core wire 32 and shaft 34 moves the ends of braid 36 towards each other, forcing the middle region of braid 36 to expand. To collapse braid 36, core wire 32 is pushed and shaft 34 is pulled, as shown by arrows 33 and 35 respectively in FIG. 3. This reverse manipulation draws the ends of braid 36 apart, pulling the middle region of braid 36 radially inward toward core wire 32.

Referring now to FIG. 5, in a first embodiment of the invention, occluder guide wire 420 includes core wire 42 and flexible tubular tip member 43, such as a coil spring, fixed around the distal end of core wire 42. Thin wires made from stainless steel and/or one of various alloys of platinum are commonly used to make coil springs for such use in guide wires. Core wire 42 can be made from shape memory metal such as nitinol, or a stainless steel wire, and is typically tapered at its distal end. The proximal end of core wire 42 may also be surrounded by a fully bonded coil, which can act as a handle and can help to prevent accidental kinking of the enclosed portion of core wire 42. Tubular shaft 444 is slidably disposed around core wire 42, and includes shaft proximal portion 446 and shaft distal portion 448. Proximal portion 446 is relatively stiff and can be made from thin walled stainless steel tubing, usually referred to as hypotubing, although other metals or rigid polymeric materials can be used. Distal portion 448 is relatively flexible and comprises coiled filament 447 surrounded by sleeve 449. Coiled filament 447 may be made from a plastic filament or metal wire, and its coils are closed, or “stacked” to enhance direct transmission of push force. Sleeve 449 may be made of thin-walled heat-shrink tubing, such as polyethylene terephthalate that has been extruded and blow-molded, or stretch blow-molded, to a larger diameter. Sleeve 449 may be heat-shrunk around coiled filament 447, and optionally, around the distal end of proximal shaft portion 446 to form a joint between proximal and distal portions 446, 448.

The length of shaft distal portion 448 may be selected as appropriate for the intended use of the occluder guide wire. In one example, distal portion 448 may be designed and intended to be flexible enough to negotiate tortuous coronary arteries, in which case the length of distal portion 448 may be 15-35 cm (5.9-13.8 inches), and preferably at least approximately 25 cm (9.8 inches). When occluder guide wire 420 is designed for use in small vessels, shaft 444 may have an outer diameter of about 0.36 mm (0.014 inch). It is advantageous for shaft distal portion 448 to extend proximally from the region of treatment site 15, out of coronary arteries 17, and over the patient's aortic arch. As can be seen in FIG. 2, such a construction would make it unnecessary for relatively stiff, proximal shaft portion 446 to extend from nearly straight descending aorta DA into the comparatively curved aortic arch. In this way, occluder guide wire 420 can have flexible distal portion 448 disposed within tortuous anatomy, while stiff proximal portion 446 can be disposed within linear anatomy. In comparison to treatment of coronary vessels, adaptations of the invention for treatment of renal arteries may require a relatively shorter flexible portion 448, and versions intended for approaching vessels in the head and neck may require a relatively longer flexible portion 448.

Expandable occluder 425 is positioned concentrically with core wire 42, and is sized such that when it is fully deployed, as shown in FIGS. 1 and 2, the outer perimeter of occluder 425 will contact the inner surface of the vessel wall. The surface contact is preferably maintained around the entire vessel lumen to prevent any atheroemboli from slipping past occluder 425. Preferably, cyanoacrylate adhesive is used to secure occluder distal end 427 to tip member 43, and to secure filter proximal end 429 near the distal end of shaft 444. Suitable cyanoacrylate instant adhesives may be obtained from Loctite Corporation, Rocky Hill, Conn., U.S.A., or Dymax Corporation, Torrington, Conn., U.S.A. Optionally, radiopaque marker bands (not shown), such as platinum rings, can be incorporated into the adhesive joints securing occluder ends 427, 429 respectively to tip member 43 and shaft 444. Occluder 425 is deployed by advancing, or pushing shaft 444 relative to core wire 42 such that occluder distal and proximal ends 427, 429 are forced toward each other, causing the middle, or central section of filter 425 to expand radially. Occluder 425 is collapsed by withdrawing, or pulling shaft 444 relative to core wire 42 such that occluder distal and proximal ends 427, 429 are drawn apart from each other, forcing the middle, or central section of occluder 425 to contract radially.

When occluder 425 transforms between a collapsed and an expanded configuration, its interior volume changes, thus potentially creating vacuum or pressure, which may inhibit the desired shape transformation of the protection element. To permit ingress and egress of fluid to and from the interior of occluder 425, non-porous membrane 428 can be fitted over braided filaments 426 such that opening 424 is formed adjacent occluder distal end 427. During shape transformation of occluder 425, fluid, such as blood can flow through opening 424 and through pores formed in braided filaments 426.

Occluder 425 also comprises open coil 445, which surrounds core wire 42 within occluder 425. Open coil 445 may be an expanded, distal extension of coiled filament 447, or it may be formed separately, of filamentous material that is similar to or different from the material of coiled filament 447. Open coil 445 may be designed as a compression spring, to assist in collapsing occluder 425. For example, open coil 445 can have a relaxed length that is longer than the collapsed length of occluder 425. In this case, transforming occluder 425 into its expanded configuration will also shorten and compress open coil 445. When acting as a compression spring, open coil 445 will be retained between the distal end of coiled filament 447 and the proximal end of tip member 43, such that it does not need to be fixedly coupled at its ends. Alternatively, open coil 445 may be designed as a tension spring, to assist in expanding occluder 425. In this example, open coil can have a relaxed length that is shorter than the length of occluder 425, when expanded into apposition with the vessel wall. To perform as a tension spring, open coil 445 must be fixedly coupled at its distal and proximal ends within occluder distal end 427 and occluder proximal end 429, respectively.

FIG. 6 depicts filter guide wire 520, a second embodiment of the invention, and in which core wire 542 comprises elongate proximal segment 590, distal segment 591, and tip member 43 mounted around the tip of distal segment 591. The length of core wire distal segment 591 may be selected as appropriate for the intended use of the filter guide wire. In one example, core wire distal segment 591 may be designed and intended to be flexible enough to negotiate tortuous coronary arteries, in which case the length of distal segment 591 maybe 15-35 cm (5.9-13.8 inches), and preferably at least approximately 25 cm (9.8 inches). When filter guide wire 520 is designed for use in small vessels, core wire proximal segment 590 may have a diameter of about 0.36 mm (0.014 inch), and core wire distal segment 591 may have a diameter of about 0.18 mm (0.007 inch).

As described above, in regard to occluder guide wire 420, it is advantageous for core wire distal segment 591 to extend proximally from the region of treatment site 15, out of coronary arteries 17, and over the patient's aortic arch, thus making it unnecessary for relatively stiff, core wire proximal segment 590 to extend from nearly straight descending aorta DA into the comparatively curved aortic arch. In this way, core wire 542 of filter guide wire 520 can have flexible distal segment 591 disposed within tortuous anatomy, while stiff proximal segment 590 can be disposed within linear vascular anatomy.

Actuator sleeve 563 is slidably disposed along core wire distal segment 591, proximal to tip member 43, and may comprise thin-walled metal or polymer tubing having an outer diameter substantially equal to the diameter of core wire proximal segment 590. Suitable metal tubing may be made of stainless steel or nitinol (NiTi), and one example of suitable polymer tubing is thermoset polyimide (PI) tubing.

Self-expanding filter 25 is mounted about core wire 542, with filter distal end 27 being coupled to tip member 43 and proximal end 29 being coupled adjacent the distal end of actuator sleeve 563. One or more inlet openings 66 are formed in filter 25 adjacent proximal end 29. Filter distal and proximal ends 27, 29 are attached using adhesive or solder, and are fitted with radiopaque markers. Proximal movement of actuator sleeve 563 along distal segment 591 will separate filter distal and proximal ends 27, 29, causing transformation of filter 25 from its self-expanded configuration to its collapsed configuration.

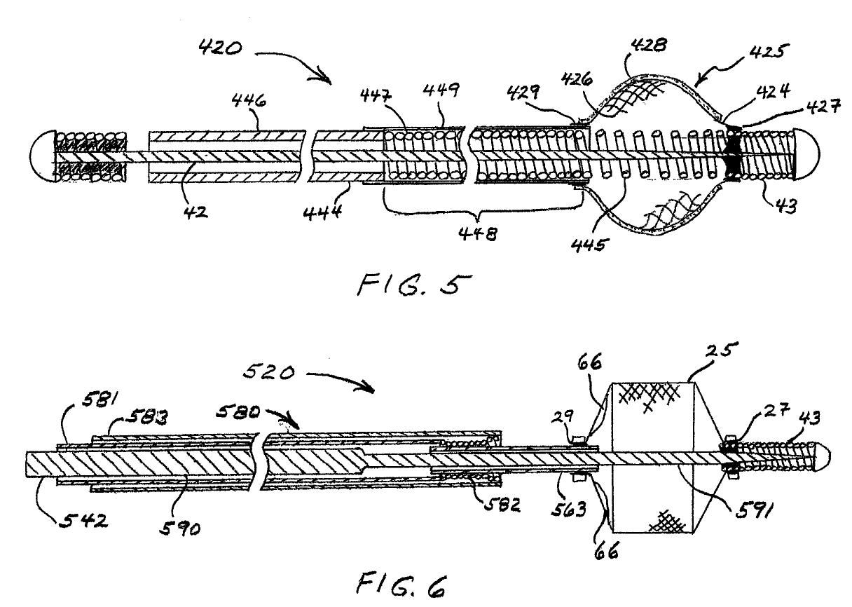

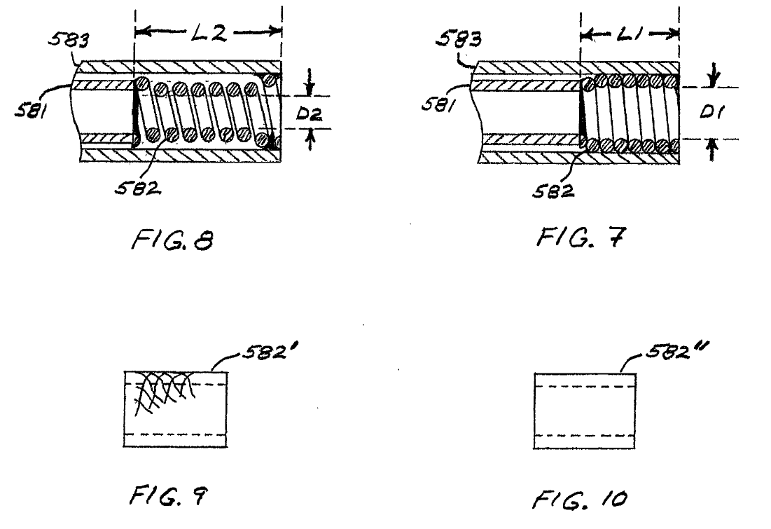

Actuator sleeve 563 can be axially manipulated from the proximal end of filter guide wire 520 by elongate operator rod 580, which is slidably and removably disposed along guide wire 520. Gripping element 582 is located within the distal end of operator rod 580 for engagement with actuator sleeve 563, as shown in FIG. 6. FIGS. 7 and 8 depict gripping element 582 in open and closed configurations, and with the gripping element proximal end coupled to the distal end of inner tube 581 and the gripping element distal end coupled to the distal end of outer tube 583. Relative displacement of inner and outer tubes 581, 583 can apply tension or compression to gripping element 582, which is a stretchable sheath having a diameter that is inversely proportional to its length. Accordingly, stretching gripping element 582 such that first length L1 extends to second length L2 causes first diameter D1 to constrict to second diameter D2.

Gripping element 582 may be relaxed in the open configuration shown in FIG. 7, which would require pulling inner tube 581 and pushing outer tube 583 to constrict first diameter D1 towards second diameter D2, thereby causing gripping element 582 to engage with actuator sleeve 563, as shown in FIG. 6. Alternatively, gripping element 582 may be relaxed in the closed configuration shown in FIG. 8, which would require pushing inner tube 581 and pulling outer tube 583 to expand second diameter D2 towards first diameter D1, thereby releasing gripping element 582 from its automatic engagement with actuator sleeve 563.

Inner and outer tubes 581, 583 may be made from metal such as stainless steel or nitinol, or from a rigid polymer such as thermoset polyimide. Gripping element 582 is shown as a coiled filament, such as a metal or plastic spring. Alternate gripping element 582′, shown in FIG. 9, comprises a segment of braided tube, which may be fabricated of metal orplastic filaments. Another alternate gripping element 582″, shown in FIG. 10, may be an elastic tube formed of materials such as natural or synthetic rubber or a thermoplastic elastomer.

FIG. 11 illustrates alternate operator rod 580′ having a locking mechanism to hold inner and outer tubes 581′, 583′ in a selected position, as may be advantageous for maintaining filter 25 in a collapsed configuration while inserting and advancing filter guide wire 520 into and through a patient's vasculature, as described below. The locking mechanism of alternate operator rod 580′ comprises pin 560′ on inner tube 581′ and slot 561′ in outer tube 583′ in an arrangement typical of a bayonet mount. Pin 560′ moves axially in slot 561′ while inner and outer tubes 581′, 583′ move with respect to each other. Rotating inner tube 581′ within outer tube 583′ can move pin 560′ into at least one locking position of slot 561′.

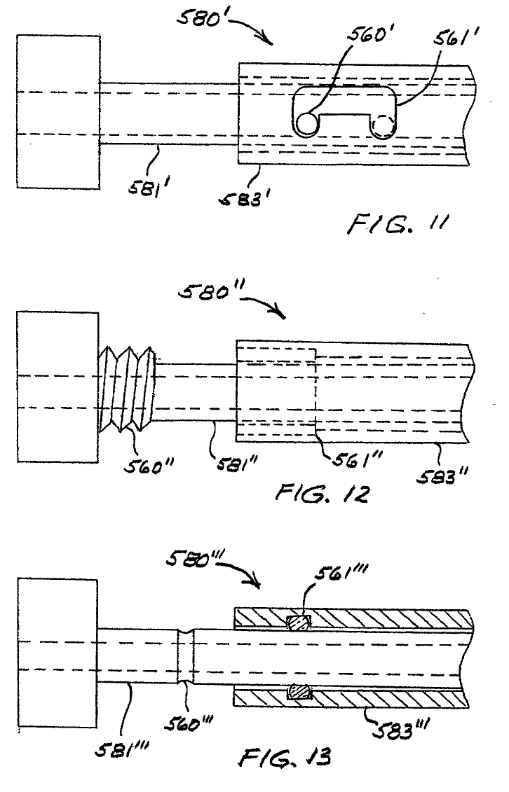

FIG. 12 illustrates alternate operator rod 580″ having an alternate locking mechanism comprising male threaded portion 560″ on inner tube 581″ and mating female threaded portion 561″ in outer tube 583″. When inner tube 581″ has been advanced sufficiently into outer tube 583″, then mating threaded portions 560″, 561″ can be screwed together. FIG. 13 illustrates alternate operator rod 580′″ having another alternate locking mechanism comprising annular groove 560′″ on inner tube 581′″ and mating elastic ring 561′″ mounted within a retention groove in outer tube 583′″. When inner tube 581′″ has been advanced sufficiently into outer tube 583′″, then elastic ring 561′″ can engage annular groove 560′″. Alternatively, instead of elastic ring 561′″, a single dimple or inner ridge may be formed in outer tube 583′″. As shown in FIGS. 11-13, inner tube 581 or its alternative embodiments may have an enlarged knob or handle at the proximal end.

In use, filter guide wire 520 may be prepared for insertion into the patient by manipulating operator rod 580, thus engaging gripping element 582 with and pulling back on actuator sleeve 563, thereby collapsing self-expanding filter 25. After inserting filter guide wire 520 through the patient's vasculature to the desired location, gripping element 582 may be released, allowing filter 25 to expand into apposition with the vessel wall. Operator rod 580 may then be withdrawn from the patient, exposing indwelling filter guide wire 520, which may then be used to guide interventional catheter 10 to treatment site 15. After completion of the therapy, catheter 10 maybe removed, followed by the use of operator rod 580 to collapse filter 25 and remove filter guide wire 520 with any atheroembolic debris retained there within.

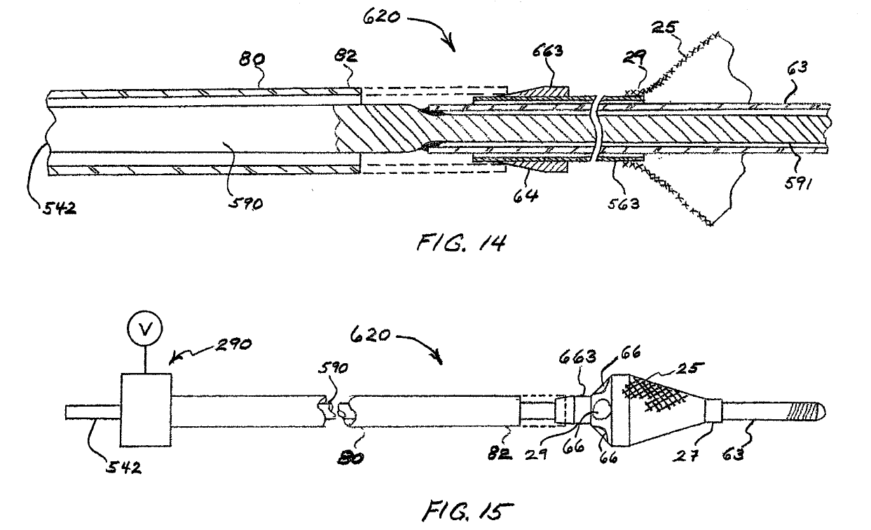

FIGS. 14 an 15 depict filter guide wire 620, a third embodiment of the invention. Filter guide wire 620 incorporates structure and components similar to those of filter guide wire 520 except that actuator 663, having proximal taper 64, is incorporated into actuator sleeve 563 to provide a conical location for releasable engagement with hollow rod 80. As shown in FIG. 14, tip member 63 may extend substantially over the length of core wire distal segment 591. Filter guide wire 620 may include, optionally, an assist spring (not shown), which is preferably a coiled tension spring mounted around guide wire 542 inside filter 25, and having distal and proximal ends fixedly coupled to filter distal and proximal ends 27, 29, respectively. The assist spring can assist in the deployment of filter 25 by providing tension between filter distal and proximal ends 27, 29. Elongate hollow rod 80 is slidably and removably disposed along guide wire 542 such that rod distal end 82 is engageable with actuator 663, as shown in the alternate positions in FIGS. 14 and 15. In similar fashion to operator rod 580, hollow rod 80 is intended to transform filter 25 from an expanded configuration to a collapsed configuration, and hollow rod 80 can be made from metal such as stainless steel or nitinol, or preferably from a rigid polymer such as thermoset polyimide.

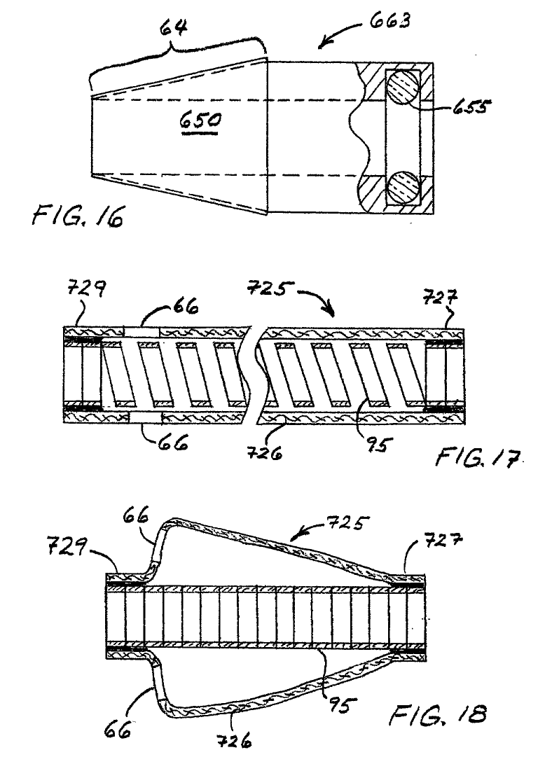

Vacuum apparatus 290, shown schematically in FIG. 15, applies a partial vacuum between hollow rod 80 and core wire 542 to form an attachment between rod distal end 82 and actuator 663. By slowly releasing the partial vacuum applied by apparatus 290, actuator 663 can be slowly released from its attachment to rod distal end 82, thus slowing the deployment of filter 25. FIG. 16 illustrates actuator 663 with two features that minimize the aspiration of fluid, such as blood into hollow rod 80, when partial vacuum is applied there through. First, coating 650 is applied over proximal taper 64 to provide a sealing surface for engagement with rod distal end 82. A hydrophilic coating or other soft material may serve as coating 650. Second, actuator 663 may also have fluid seal 655 for slidably sealing around core wire 542. Fluid seal 655 may comprise an o-ring, as shown in FIG. 16, or any other functional configuration, such as a slit diaphragm or gasket. In order for fluid seal 655 to maintain sealing contact with core wire 542, filter guide wire 560 would not include actuator sleeve 563. Rather, filter proximal end 29 would be directly affixed to actuator 663, as shown in FIG. 15.

FIGS. 17-18 illustrate self-closing filter 725, a fourth embodiment of the invention. Filter 725 comprises a tube formed by braided filaments 726 that define pores, and at least one inlet opening 66 that is substantially larger than the pores. An alternative filter structure may employ braided filaments 726 to provide support for a porous membrane, in which case the size of the filter pores is defined by the porous membrane rather than by braid filaments 726. Other types of assemblies that may be used in filter 725 include an expandable strut structure (not shown), which may be made from a slotted or slit tube, covered with either a non-porous membrane (not shown) to provide an occluder, or a porous membrane to provide a filter.

Incorporated into filter 725 is spring 95, which is a coiled compression spring disposed within filter 725 and having a distal end affixed to filter distal end 727 and having a proximal end affixed to filter proximal end 729. Spring 95 assists in the collapse of filter 725 by providing separating force between filter distal and proximal ends 727, 729. Assist spring 95 can be fabricated with flat wire, or ribbon, as shown in FIGS. 17 and 18, or with fine metal wire of about 0.03 to 0.13 mm (0.001 to 0.005 inch) diameter, preferably nitinol wire having 0.08 mm (0.003 inch) diameter.

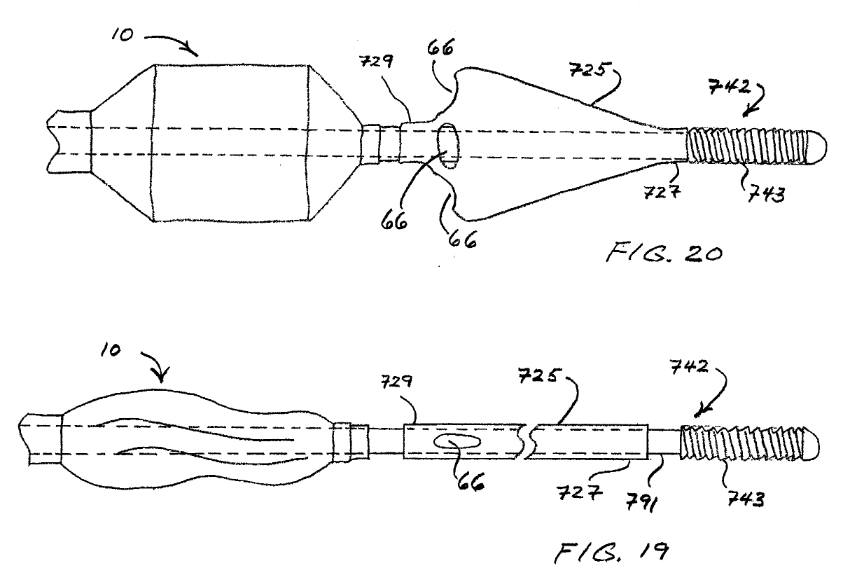

As shown in FIGS. 19 and 20, filter 725 can be selectively loaded onto the proximal end of core wire 742 and slidably advanced there along to a desired location within core wire distal region 791. Filter distal end 727 can be blocked from further distal advancement by a stop element such as tip member 743. In an alternative embodiment, filter 725 can be permanently pre-mounted to core wire 742 by having filter distal end 727 fastened to core wire distal region 791, preferably, with cyanoacrylate adhesive. An elongate operator, such as a hollow push rod or catheter 10 can be advanced over core wire 742 to abut filter proximal end 729 to deploy self-collapsing filter 725. Distally pushing catheter 10, while proximally pulling core wire 742 will transform filter 725 by overcoming the shape memory of filter 725 and/or the compression force in spring 95, as depicted in FIG. 20.

The methods of using of guide wire apparatuses of the invention will be described below. In one example, filter guide wire 520, having self-expanding filter 25 and actuator 563, is provided. Operator rod 580 is advanced over core wire 542 and gripping element 582 is manipulated to engage and proximally withdraw actuator 563 along core wire 542, thereby collapsing filter 25. With filter 25 in the collapsed configuration, filter guide wire 520 and operator rod 580 are advanced into the patient's vasculature until filter 25 is distal to the intended treatment site. Disengagement of gripping element 582 and withdrawal of operator rod 580 allows filter 25 to expand under its own shape memory. With filter 25 deployed into contact with the vessel wall, a therapeutic catheter is advanced over filter guide wire 520 to the intended treatment site, and the therapy, such as balloon angioplasty, is performed. Any atheroembolic debris generated during the therapy is captured in filter 25. After the therapy is completed, the therapeutic catheter is withdrawn. As described above, operator rod 580 is again used to collapse filter 25. With filter 25 in the collapsed configuration, filter guide wire 520 and operator rod 580 are withdrawn from the patient's vasculature.

While the invention has been particularly shown and described with reference to the preferred embodiments thereof, it will be understood by those skilled in the art that various changes in form and detail may be made therein without departing from the spirit and scope of the invention. For example, the invention may be used in any intravascular treatment utilizing a guide wire where the possibility of loosening emboli may occur. Although the description herein illustrates angioplasty and stent placement procedures as significant applications, it should be understood that the present invention is in no way limited to those environments.

What is claimed is:

1. A guide wire apparatus comprising:
an elongate core wire having proximal and distal ends;
a flexible tubular tip member fixed adjacent the core wire distal end;
an elongate tubular shaft slidably disposed along the core wire, the shaft including:
an elongate proximal portion having a relatively stiff, solid wall and comprising a majority of the length of the tubular shaft;
a relatively short distal portion comprising a coiled filament, the distal portion being more flexible than the proximal portion and being disposed proximally of the tip member; and
a generally tubular, self-collapsing protection element mounted coaxially about the core wire, the protection element having a distal end fixed to the tip member and a proximal end fixed to the shaft distal portion, wherein relative displacement of the filter ends associated with longitudinal displacement between the tubular shaft and the core wire causes transformation of the protection element between a collapsed configuration and an expanded configuration.

2. The guide wire apparatus of claim 1 wherein the tubular shaft proximal portion comprises metal tubing.

3. The guide wire apparatus of claim 1 wherein the coiled filament comprises metal wire.

4. The guide wire apparatus of claim 1 wherein the shaft distal portion extends proximally from a location adjacent the tip member, through the protection element to a distal end of the shaft proximal portion.

5. The guide wire apparatus of claim 4 wherein a segment of the shaft distal portion extending through the protection element comprises an open-coiled filament.

6. The guide wire apparatus of claim 5 wherein a segment of the shaft distal portion extending proximally from the protection element comprises a closed-coiled filament.

7. The guide wire apparatus of claim 6 wherein the closed-coil filament is covered by a polymer sleeve.

8. The guide wire apparatus of claim 7 wherein the polymer sleeve comprises polyethylene terephthalate heat-shrink tubing.

9. The guide wire apparatus of claim 7 wherein the polymer sleeve extends over the shaft proximal portion by a sufficient distance to effect a joint between the shaft proximal and distal portions.

10. The guide wire apparatus of claim 1 wherein the protection element is a filter.

11. The guide wire apparatus of claim 1 wherein the protection element is an occluder.

12. A guide wire apparatus comprising:
an elongate flexible core wire having a proximal end and a flexible tip member fixed about a core wire distal region;
a generally tubular, self-expanding protection element mounted coaxially about the core wire distal region, the protection element having a distal end fixedly coupled to the tip member and a proximal end slidingly disposed about the core wire distal region, wherein relative axial separation of the ends of the protection element causes transformation of the protection element from an expanded configuration to a collapsed configuration;
an actuator sleeve having proximal and distal ends and being slidably disposed about the core wire distal region, the sleeve distal end being coupled to the proximal end of the protection element; and
an elongate operator rod having proximal and distal ends and being slidably and removably disposed about the core wire, the rod comprising inner and outer tubes being axially moveable with respect to each other to operate a gripping mechanism disposed within the rod distal end, the gripping mechanism being releasably engageable with the sleeve proximal end to draw the sleeve proximally over the core wire, thus transforming the protection element from the expanded configuration to the collapsed configuration.

13. The guide wire apparatus of claim 12 wherein the gripping mechanism comprises a stretchable sheath having a diameter that is inversely proportional to its length, and having proximal and distal ends attached to distal ends of the rod inner and outer tubes, respectively.

14. The guide wire apparatus of claim 13 wherein the stretchable sheath has a relaxed inner diameter that is less than an outer diameter of the actuator sleeve.

15. The guide wire apparatus of claim 13 wherein the stretchable sheath has a relaxed inner diameter that is greater than an outer diameter of the actuator sleeve.

16. The guide wire apparatus of claim 13 wherein the stretchable sheath is selected from a group consisting of coiled springs, braided tubes, elastic tubes and elastomeric tubes.

17. The guide wire apparatus of claim 12 wherein the operator rod further comprises a locking mechanism for holding the inner and outer tubes in a selected position with respect to each other.

18. The guide wire apparatus of claim 17 wherein the locking mechanism is selected from a group consisting of detents, bayonet mounts, interference fits and screw threads.

19. The guide wire apparatus of claim 12 wherein the protection element is a filter.

20. The guide wire apparatus of claim 12 wherein the protection element is an occluder.

21. A guide wire apparatus comprising:
an elongate flexible core wire having a proximal end, a distal region, and a flexible tubular element fixed about the core wire distal region;
a generally tubular, self-expanding protection element mounted coaxially about the core wire, the protection element having distal and proximal ends slidingly disposed about the core wire, wherein relative longitudinal movement between the distal and proximal ends of the protection element accompanies a transformation of the protection element between a closed configuration and an expanded configuration;
a tubular actuator fixed to the proximal end of the protection element and being slidably disposed about the core wire, the actuator having a proximal tapered surface and a fluid seal capable of slidingly sealing around the core wire; and
an elongate hollow rod having a lumen and a distal end releasably engageable with the actuator by applying a partial vacuum through the lumen of the hollow rod, the hollow rod being slidably disposed about the core wire and being operable, when the rod distal end is engaged with the actuator, to draw the actuator proximally over the core wire, thus transforming the protection element from the expanded configuration to the collapsed configuration.

22. The guide wire apparatus of claim 21 wherein the tubular actuator further comprises a coating on the proximal tapered surface for enhancing a vacuum seal between the tapered surface and the rod distal end.

23. The guide wire apparatus of claim 22 wherein the coating comprises a hydrophilic material.

24. The guide wire apparatus of claim 21 wherein the protection element is a filter.

25. The guide wire apparatus of claim 21 wherein the protection element is an occluder.

26. An apparatus for prevention of distal atheroembolization, the apparatus comprising:
a tubular protection element having distal and proximal ends, wherein relative longitudinal movement between the distal and proximal ends of the protection element accompanies a transformation of the protection element between a closed configuration and an expanded configuration; and
a coiled compression spring disposed within the tubular protection element and having distal and proximal ends coupled to the distal and proximal ends of the protection element, respectively, such that the compression spring urges the tubular protection element to remain in the closed configuration, the compression spring being sized and shaped to fit slidably over a core wire.

27. The apparatus of claim 26 further comprising a core wire slidably disposed within the protection element and the compression spring, the core wire having a distal end and a tip member fixedly mounted adjacent thereto, the tip member limiting distal advancement of the protection element and the compression spring along the core wire.

28. The apparatus of claim 27 further comprising an elongate, hollow, deployment rod slidably and removably disposed about the core wire, the deployment rod being operable to push the protection element distally along the core wire until the protection element is longitudinally compressed between the deployment rod and the tip member, thereby effectuating the transformation of the protection element from the closed configuration to the expanded configuration.

29. The apparatus of claim 28 wherein the deployment rod comprises an interventional catheter.

30. The apparatus of claim 26 wherein the protection element is a filter.

31. The apparatus of claim 26 wherein the protection element is an occluder.
